(19) United States
(12) Patent Application Publication
(10) Pub. No.: US 2020/0022936 A1
(43) Pub. Date: Jan. 23, 2020

(54) COMBINATIONS OF DICLOFENAC, H2 RECEPTOR ANTAGONISTS AND ALKALI METAL BICARBONATES FOR THE TREATMENT OF PAIN AND INFLAMMATION

(71) Applicant: Mehmet Nevzat Pisak, Besiktas/Istanbul (TR)

(72) Inventor: Mehmet Nevzat Pisak, Besiktas/Istanbul (TR)

(21) Appl. No.: 16/497,571

(22) PCT Filed: Nov. 16, 2018
(86) PCT No.: PCT/TR2018/050700

§ 371 Date: Sep. 25, 2019

(30) Foreign Application Priority Data
Nov. 16, 2017 (TR) 2017/18099

(51) Int. Cl. A61K 31/196; A61K 31/426

(52) U.S. Cl. CPC A61K 31/196; A61K 9/0007; A61K 31/426

(57) ABSTRACT

A combination comprising: a) diclofenac or a pharmaceutically acceptable salt thereof, b) an H2 receptor antagonist, preferably famotidine or a pharmaceutically acceptable salt thereof, and c) an alkali metal carbonate or bicarbonate, preferably potassium bicarbonate for use in the treatment of pain and inflammation.

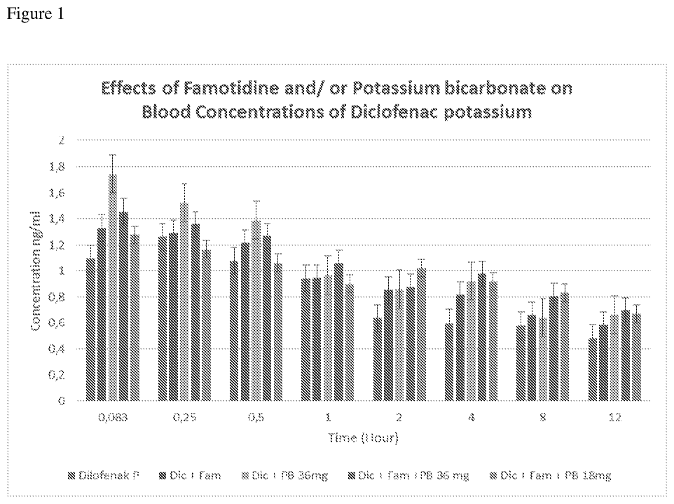

### TECHNICAL FIELD

The present invention relates to pharmaceutical combinations comprising diclofenac, an H2 receptor antagonist preferably famotidine and an alkali metal bicarbonate, to oral dosage forms comprising such combinations and to processes for the preparation thereof.

### BACKGROUND ART

Diclofenac is a non-steroidal anti-inflammatory drug (NSAID) and has been widely prescribed for the treatment of pain and inflammation. However it is well known that it has the potential to cause gastrointestinal (GI) side effects, such as gastric and duodenal ulcers, bleeding and perforation, oesophageal inflammation and strictures, and small bowel and colonic ulcers and strictures.

Laine L. (Semin. Arthritis Rheumatism. 2002; 32:25-32) reports that NSAIDs exert their pharmacological action by inhibiting the synthesis of prostaglandins (PGs) by non-selectively blocking cyclooxygenases 1 and 2 (COX-1 and COX-2) or by selectively blocking COX-2. Inhibition of COX-1 is also responsible, in part, for gastrointestinal side effects, which are the most frequent side effects of NSAIDs.

Gwaltney-Brant S. M. reports that non-selective COX inhibitors have other contributors to their gastrointestinal side effects, which are the carboxylic acid group in compounds, such as aspirin, ibuprofen and diclofenac, and the acidic enolic group in oxicams, such as piroxicam (Charlene A. M., editor. Comprehensive Toxicology. 2nd ed. Elsevier; Oxford, UK: 2010. pp. 159-161). These acidic groups cause local irritation upon oral administration, which can lead to the clinically observed gastrointestinal side effects either independently or in tandem with inhibition of the COX-1 enzyme.

In a report, Lanas et al. (2011) have concluded that more than 90% of the treated patients with osteoarthritis are at increased GI risk, with 60% of them at high risk.

Another important issue related to the use of NSAIDS, in particular diclofenac is the time of onset and duration of action. It is desirable to obtain a rapid onset of action and long duration of analgesic effect for an efficient pain management.

Diclofenac is a proven, commonly prescribed NSAID that has analgesic, anti-inflammatory, and antipyretic properties, and has been shown to be effective in treating a variety of acute and chronic pain and inflammatory conditions.

Vane J R. et al. (Nat N Biol. 1971; 231(25):232-5), Ku E C. et al. (Am J Med. 1986; 80(4B):18-23), and Patrono C. et al. (J Clin Invest. 2001; 108(1):7-13) report that diclofenac belongs to a group of NSAIDs that inhibit both COX-1 and COX-2 enzymes. The binding of NSAIDs to COX isozymes inhibits the synthesis of prostanoids (i.e., prostaglandin [PG]-E2, PGD2, PGF2, prostacyclin [PGI2], and thromboxane [TX] A2).

Furthermore, Patrono C. et al. (J Clin Invest. 2001; 108(1):7-13), Smyth E M et al., and Grosser T. et al. (J Clin Invest. 2006; 116(1):4-15) report that PGE2 is the dominant prostanoid produced in inflammation, and the inhibition of its synthesis by NSAIDs is believed to be the main mechanism of the potent analgesic and anti-inflammatory properties of these agents.

Diclofenac, similar to other NSAIDs, is associated with an increased risk of serious dose-related GI side effects.

Since its introduction in 1973, a number of different diclofenac-containing drug products have been developed with the goal of improving efficacy, tolerability, and patient convenience.

U.S. Pat. No. 7,482,377 B2 discloses a unit dose combination of diclofenac and alkali metal bicarbonates for a faster onset of action, although this particular combination does provide a solution for fast pain relief, it does not address the issue of GI side effects and pain relief over extended periods of time.

U.S. Pat. No. 8,946,292 B2 describes new formulations of diclofenac with hydroxypropyl beta cyclodextrin claiming that the efficacy of diclofenac solubilized with hydroxypropyl-beta-cyclodextrin at several dose levels suggests a faster onset of action. Although this creates an interesting treatment modality for an injectable preparation, it does not have the synergistic effect that H2 receptor antagonists and alkali metal bicarbonates have together when administered with diclofenac. Furthermore, the methods and formulations of the present invention have the added benefit of oral self administration and diminished GI side effects.

Therefore a particular need exists for a composition giving fast and long duration of pain relief with decreased side effects.

The aim of this invention is to develop a combination comprising diclofenac with one or more agents to reduce the occurence of gastro-intestinal side effects and at the same time providing an efficient pain management.

### SUMMARY OF THE INVENTION

The present invention provides a combination comprising diclofenac or a pharmaceutically acceptable salt thereof, an H2 receptor antagonist, or a pharmaceutically acceptable salt thereof and an alkali metal bicarbonate. The pharmaceutical composition comprising said three active ingredients provides numerous advantages over either ingredient alone or combinations containing only two of them, including: a significant reduction in the time of onset of action, pain relief over extended periods of time and reduced side effects.

In one embodiment, pharmaceutical combination of the present invention comprises diclofenac, or a pharmaceutically acceptable salt thereof, an H2 receptor antagonist, selected from the group consisting of famotidine, pabutidine, lafutidine, loxtidine, nizatidine, roxatidine, tiotidine, niperotidineandoxmetidine, or pharmaceutically acceptable salts thereof; famotidine being the preferred H2 receptor antagonist, and an alkali metal bicarbonate.

In one embodiment, pharmaceutical combination of the present invention comprises diclofenac, or a pharmaceutically acceptable salt thereof, an H2 receptor antagonist, or a pharmaceutically acceptable salt thereof and an alkali metal carbonate or alkali metal bicarbonate, selected from the group consisting of potassium bicarbonate, sodium bicarbonate, ammonium bicarbonate and calcium bicarbonate; potassium bicarbonate being the preferred one.

In another embodiment, pharmaceutical combination of the present invention comprises diclofenac, famotidine and potassium bicarbonate.

Furthermore, the present invention provides the use of a combination of diclofenac, an H2 receptor antagonist and an alkali metal bicarbonate for the treatment of inflammation and pain. In one embodiment said combination is used for the treatment of inflammation and pain caused by muscular or skeletal system diseases. In another embodiment said combination is used for the treatment of inflammation and pain caused by chronic polyarthritis, ankylosing spondilytis, osteoarthritis, gout attacks, extra-articular rheumatism, post-traumatic and postoperative pain, or dysmenorrhea.

The present invention further relates to pharmaceutical oral dosage forms comprising a combination of disclofenac, an H2 receptor antagonist and an alkali metal bicarbonate and at least one pharmaceutically acceptable carrier.

### DETAILED DESCRIPTION OF THE INVENTION

The present invention provides a combination comprising a) diclofenac, or a pharmaceutically acceptable salt thereof, b) an H2 receptor antagonist, or a pharmaceutically acceptable salt thereof and c) an alkali metal carbonate or bicarbonate.

The combination of an H2 receptor antagonist with diclofenac has the benefit of diminishing the gastro-intestinal side effects associated with diclofenac, and creates a treatment that can be used for longer periods of time due to the decreased side effects.

Furthermore, the combination of an H2 receptor antagonist, preferably famotidine with diclofenac, surprisingly increases the permeability and rate of absorption of the diclofenac. In other words, said combination increases Cmax and AUC values of diclofenac and decreases the Tmax.

The addition of an alkali metal bicarbonate to the combination of H2 receptor antagonist and diclofenac has added further synergistic effect, whereby higher plasma levels of diclofenac is obtained in a shorter period of time compared to the combination of H2 receptor antagonist and diclofenac. Furthermore, the Cmax and AUC of diclofenac have increased compared to the combination of two active ingredients, namely famotidine (an H2 receptor antagonist) and diclofenac. Thus the preferred combination for the treatment of pain and inflammation comprises an alkali metal bicarbonate in addition to a H2 receptor antagonist and diclofenac.

For the purposes of the present invention Tmax means the amount of time that a drug takes to reach the peak concentration in serum; Cmax is the peak serum concentration of a drug; and AUC (the area under the curve) represents the area under the plasma concentration curve, also called the plasma concentration-time profile, a measure of total systemic exposure to the drug.

In one embodiment of the present invention combination comprises potassium or sodium salt of diclofenac, most preferably potassium salt due to the fact that potassium salt is associated with a faster absorption and as a result of this a more rapid onset of pain relief is achieved compared to the sodium salt. On the other hand the potassium salt still has, at least the same GI side effects and still does not have a fast enough onset of action.

The chemical structure of diclofenac is shown in Formula 1.

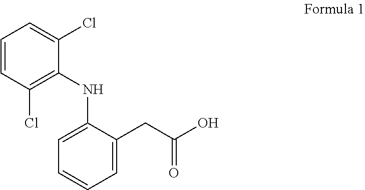 [SMILES: O=C(O)Cc1ccccc1Nc1c(Cl)cccc1Cl]

The chemical name of diclofenac is 2[(2,6-dichlorophenyl)amino]benzeneacetic acid.

In one embodiment of the present invention combination comprises famotidine, or pharmaceutically acceptable salt thereof as an H2 receptor antagonist.

Famotidine protects the gastric mucosa against irritation, thus it is used in the treatment of gastrointestinal diseases. Its chemical structure is shown in Formula 2.

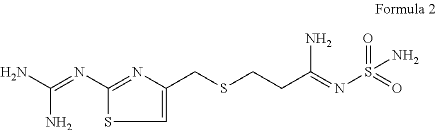 [SMILES: NC(N)=Nc1nc(CSCC/C(N)=N/S(N)(=O)=O)cs1]

The chemical name of famotidine is 3-(2-(diaminomcthyleneamino)thiazol-4-ylmethylthio)-N-sulfamoylpropionamidine.

In one embodiment of the present invention combination comprises potassium bicarbonate as alkali metal bicarbonate.

Potassium bicarbonate (also known as potassium hydrogen carbonate or potassium acid carbonate) is a colorless, odorless, slightly basic, salty substance used to neutralize acid in the stomach.

In one embodiment of the present invention the combination comprises diclofenac, famotidine and an alkali metal bicarbonate. It has now been found that said combination provides the most efficient diclofenac treatment when a fast onset of action and over an extended period of time pain reduction is needed. The reason why this combination can be stated as the most efficient diclofenac treatment is that the results obtained in the current study demonstrate a superior, surprising effect with a Cmax over 1400 ng/ml, a Tmax below 10 minutes and an AUC over 10.000 ng·h/ml·in a 12 hour period. These results clearly demonstrate the synergistic effect between alkali metal bicarbonates, H2 receptor antagonists and diclofenac. Thus the present invention provides a gastroprotective, antiarthritic/analgesic combination with fast and extended period of time pain relief and reduced risk of developing gastrointestinal damage such as ulcers, erosions and hemorrhages. In other words, the present invention is able to provide a longer period of therapeutic effect due to the slow elimination of diclofenac as evidenced by it's high AUC value despite its instant release and fast onset of action.

The combination of the present invention is synergistically effective for the treatment of inflammation and pain by reducing gastrointestinal side effects, therefore having a prophylactic effect on the primary side effect of diclofenac administration while increasing diclofenac's Cmax and AUC and decreasing its Tmax. The single unit pharmaceutical dosage forms of the present invention would also allow for relatively safe administration of high doses of diclofenac and long administration duration, which would be especially important for patients who have acute pain attacks in a period shorter than 6 months but also for patients who have pain and inflammation related problems over a long period of time.

The combination of the present invention can be administered in various dosage forms and strength in pharmaceutically effective amount. A unit dosage form containing the combination of present invention may be in the form of a tablet, capsule, pellet, granule, effervescent tablet, tablet in tablet, tablet in capsule or powder, preferably tablet, capsule or powder form.

The pharmaceutical dosage forms of the present invention may comprise active components in the form of a racemic mixture, or in the form of substantially pure enantiomers or salts thereof.

In one embodiment of the present invention, the combination comprises between 12.5 to 100 mg, preferably 25 to 50 mg, more preferably 50 mg of diclofenac or pharmaceutically acceptable salt thereof.

In one embodiment of the present invention, the combination comprises between 10 to 60 mg, preferably 20 to 40 mg, more preferably 20 mg of famotidine or pharmaceutically acceptable salt thereof.

In one embodiment of the present invention, the combination comprises between 10 to 120 mg, preferably 20 to 80 more preferably 30 to 50 mg of potassium bicarbonate.

Further described is a pharmaceutical unit dosage form, preferably a tablet or capsule, wherein the active ingredients consist of:
a) between 12.5 to 100 mg, preferably 25 to 50 mg of diclofenac or pharmaceutically acceptable salt thereof,
b) between 10 to 60 mg, preferably 20 to 40 mg of famotidine or pharmaceutically acceptable salt thereof,
c) between 20 to 80 mg, preferably 30 to 60 mg of potassium bicarbonate.

Further described is a pharmaceutical unit dosage form, preferably a tablet or capsule, wherein the active ingredients consist of:
d) 50 mg of diclofenac or pharmaceutically acceptable salt thereof,
e) 20 mg of famotidine or pharmaceutically acceptable salt thereof,
a) 36 mg of potassium bicarbonate.

Oral dosage forms of the present invention may comprise suitable diluents, binders, lubricants, disintegrating agents, surfactants, sweetening agents, coloring agents and coating agents.

Examples of pharmaceutically acceptable diluents include, but not limited to, magnesium stearate, lactose, microcrystalline cellulose, starch, pre-gelatinized starch, calcium phosphate, calcium sulfate, calcium carbonate, mannitol, sorbitol, xylitol, sucrose, maltose, fructose and dextrose.

Examples of pharmaceutically acceptable binders include, but not limited to, starches, natural sugars, corn, sweeteners, natural and synthetic gums, cellulose derivatives, gelatin, PVP, polyethylene glycol, waxes, sodium alginate, alcohols and water.

Examples of pharmaceutically acceptable lubricants include, but not limited to, metallic stearates, metallic lauryl sulfates, fatty acids, fatty acid esters, fatty alcohols, paraffins, hydrogenated vegetable oils, polyethylene glycols, boric acid, sodium benzoate, sodium acetate, sodium chloride and talk.

Examples of pharmaceutically acceptable disintegrating agents include, but not limited to, starches, cellulose derivatives, PVP, crospovidone, clays, ion-exchange resins, alginic acid and sodium alginate.

Examples of pharmaceutically acceptable surfactants of the present invention include, but not limited to, sulfates, sulfonates, phosphates, carboxylates, primary-secondary-tertiary amines, quaternary ammonium compounds, fatty alcohols, sugar esters of fatty acids, glycerides of fatty acids, polyoxy ethylene glycol alkyl ethers, polisorbates, sorbitan alkyl esters and poloxamers.

In one embodiment of the present invention, pharmaceutical dosage form is an immediate release tablet comprising diclofenac potassium, famotidine and potassium bicarbonate as active compounds; lactose, microcrystalline cellulose, hydroxypropyl cellulose, crospovidone, colloidal silicon dioxide and magnesium stearate as inactive ingredients.

The combination of the present invention is useful for the treatment of inflammation and pain which are caused by chronic polyarthritis, ankylosing spondilytis, osteoarthritis, gout attacks, extra-articular rheumatism, post-traumatic and postoperative pain or dysmenorrheal.

In one embodiment of the present invention an oral pharmaceutical fixed-dose composition is provided which comprises the combination of a) diclofenac b) an H2 receptor antagonist, preferably famotidine or a pharmaceutically acceptable salt thereof, and c) an alkali metal carbonate or bicarbonate, preferably potassium or sodium bicarbonate, and one or more pharmaceutically acceptable excipients.

In another embodiment of the present invention, pharmaceutical composition comprises diclofenac, famotidine and potassium or sodium bicarbonate wherein the Tmax of diclofenac is less than 30 minutes, preferably less than 10 minutes.

In another embodiment of the present invention, pharmaceutical composition comprises diclofenac, famotidine and potassium or sodium bicarbonate wherein the Cmax of diclofenac is between 1200 to 1500 ng/ml, preferably over 1300 ng/ml.

In another embodiment of the present invention, pharmaceutical composition comprises diclofenac, famotidine and potassium or sodium bicarbonate wherein the AUC of diclofenac within 12 hours of administration is between 10,000 to 10,600 ng/ml·h, preferably over 9,500 ng/ml·h.

In another embodiment of the present invention, pharmaceutical composition comprises diclofenac, famotidine and potassium or sodium bicarbonate wherein famotidine and potassium or sodium bicarbonate increases the Cmax of diclofenac by at least 5%.

In another embodiment of the present invention, pharmaceutical composition comprises diclofenac, famotidine and potassium or sodium bicarbonate wherein famotidine and potassium or sodium bicarbonate creates an AUC at least 10% higher than the diclofenac administered alone in a 12 hour period.

In another embodiment of the present invention, pharmaceutical composition comprises diclofenac, famotidine and potassium or sodium bicarbonate wherein famotidine creates a Tmax of less than about 25 minutes for diclofenac.

In another embodiment of the present invention, an oral single unit pharmaceutical dosage form with an instant release profile comprises diclofenac, famotidine and potassium or sodium bicarbonate wherein at least 25% of diclofenac and at least 25% of famotidine is released in the stomach before reaching the intestines.

In another embodiment of the present invention a faster onset of pain relief or faster antipyretic/anti-inflammatory effect beginning less than 30 minutes after oral administration of the combination comprising diclofenac, famotidine and sodium or potassium bicarbonate is obtained due to the increased rate of diclofenac absorption potentiated by famotidine and also due to the synergistic, pharmacokinetic effect created by alkali metal bicarbonates and famotidine administered together with diclofenac.

In another embodiment of the present invention an oral single unit pharmaceutical dosage form comprises diclofenac, famotidine and potassium or sodium bicarbonate, wherein diclofenac combined with famotidine and alkali metal bicarbonates creates higher than usual blood concentration of diclofenac in the blood stream compared to the oral administration of diclofenac alone.

In another embodiment of the present invention administration of a single unit pharmaceutical dosage form comprises the combination of diclofenac, famotidine and potassium or sodium bicarbonate for use in the treatment of inflammation or pain caused by muscular or skeletal system diseases.

In another embodiment of the present invention administration of a single unit pharmaceutical dosage form comprises the combination of diclofenac, famotidine and potassium or sodium bicarbonate to attain fast pain or inflammation relief under 30 minutes after oral administration.

In another embodiment of the present invention administration of a single unit pharmaceutical dosage form comprises the combination of diclofenac, famotidine and potassium or sodium bicarbonate wherein inflammation or pain is caused by chronic polyarthritis, ankylosing spondilytis, osteoarthritis, gout attacks, extra-articular rheumatism, post-traumatic and postoperative pain, rheumatoid arthritis and dysmenorrheal.

In another embodiment of the present invention administration of a single unit pharmaceutical dosage form comprises the combination of diclofenac, famotidine and potassium or sodium bicarbonate to create a fast anti-pyretic effect.

In another embodiment of the present invention administration of a single unit pharmaceutical dosage form comprises the combination of diclofenac, famotidine and potassium or sodium bicarbonate wherein the oral dosage form can be administered between 1 to 4 times a day.

### Details of the Study Proving the Synergistic Effect Between Diclofenac, Famotidine and Potassium Bicarbonate

### Studying the Effects of Famotidine and Potassium Bicarbonate on the Pharmacokinetics of Diclofenac in Rats

The primary objective of the test was to compare the pharmacokinetics of diclofenac potassium when used as a single active compound with diclofenac potassium in combination with famotidine and/or potassium bicarbonate. In this experimental study, the aim was to observe how the addition of potassium bicarbonate to diclofenac and famotidine would change the pharmacokinetic properties of diclofenac potassium when diclofenac potassium is given alone or in combination with famotidine and/or potassium bicarbonate.

### Materials and Methods

### Chemicals and Reagents

Diclofenac potassium, famotidine, potassium bicarbonate.

### Animals

Male Wistar rats (240-260 g) were used in the study. The rats were maintained in an air-conditioned animals quarter at a temperature of 22±2° C. and a relative humidity of 50±10%. Food and water were allowed ad libitum. The animals were acclimatized to the facilities for five days, and then fasted with free access to water for 12 h prior to the experiment. All the animals were housed under similar conditions.

### Drug Administration

Bioavailability and pharmacokinetics of diclofenac were studied in all the normal state of rats following an oral administration of 1 mg/kg diclofenac potassium, 0.4 mg/kg famotidine and 0.72 mg/kg or 0.36 mg/kg potassium bicarbonate in different occasions. Each rat was further subjected to similar studies after administration of diclofenac potassium and/or diclofenac in combination with famotidine. A total of 30 rats were used in the study divided into 5 groups, each group was administered; diclofenac alone, or 4 different combinations of diclofenac as outlined below.

Six male and/or female rats per group were lavaged with 1 mg/kg Diclofenac potassium and combinations at a dosing volume of 2 ml/kg. Blood (0.2 ml) was taken from the tail vein prior to administration of test substances (0 h) and after 0.083, 0.167, 0.25, 0.5, 1, 2, 4, 8 and 12 h.

### Extraction of Blood Samples

Blood samples were collected in tubes containing %5 Na2-EDTA and kept on ice until 50 ul dichloromethane was added and they were centrifuged at 7000×g for 5 min at 4° C. and supernatants were collected for LC/MSMS analysis.

### Results

TABLE 1
Blood Concentrations of Diclofenac Potassium

| | Time | | | | | | | |
|---|---|---|---|---|---|---|---|---|
| Groups | 0.083 h | 0.25 h | 0.5 h | 1 h | 2 h | 4 h | 8 h | 12 h |
| Diclofenac-K | 1.096 ± 0.054 | 1.262 ± 0.048 | 1.078 ± 0.137 | 0.943 ± 0.015 | 0.600 ± 0.159 | 0.600 ± 0.015 | 0.578 ± 0.033 | 0.486 ± 0.092 |
| Dic-K + Fam | 1.331 ± 0.056 | 1.289 ± 0.041 | 1.216 ± 0.052 | 0.948 ± 0.027 | 0.851 ± 0.037 | 0.816 ± 0.023 | 0.661 ± 0.177 | 0.584 ± 0.045 |
| Dic-K + | 1.746 ± 0.22 | 1.453 ± 0.182 | 1.388 ± 0.133 | 0.967 ± 0.0181 | 0.858 ± 0.075 | 0.921 ± 0.2185 | 0.642 ± 0.076 | 0.664 ± 0.218 |
| 0.72 mg/kg | | | | | | | | |
| PB(36 mg) | | | | | | | | |
| DIc-K + Fam + | 1.457 ± 0.053 | 1.359 ± 0.099 | 1.269 ± 0.148 | 1.059 ± 0.136 | 0.875 ± 0.079 | 0.978 ± 0.13 | 0.808 ± 0.128 | 0.695 ± 0.129 |
| 0.72 mg/kg | | | | | | | | |
| PB(36 mg) | | | | | | | | |
| Dic-K + Fam + | 1.277 ± 0.075 | 1.166 ± 0.116 | 1.06 ± 0.291 | 0.901 ± 0.063 | 1.022 ± 0.067 | 0.918 ± 0.021 | 0.832 ± 0.029 | 0.672 ± 0.027 |
| 0.36 mg/kgPB | | | | | | | | |
| (18 mg) | | | | | | | | |

TABLE 2
Pharmacokinetic Parameters of Diclofenac in blood

| | | Cₘₐₓ | AUC 12 |
|---|---|---|---|
| Groups | Tₘₐₓ | (ng/ml) | hours(ng/ml · h) |
| Diclofenac-K | 0.25 | 1262 ± 0.048 | 7.5101 |
| Dic-K + Fam | 0.083 | 1331 ± 0.056 | 9.0834 |
| Dic-K + 0.72 mg/kg | 0.083 | 1746 ± 0.22 | 9.6551 |
| PB(36 mg) | | | |
| Dİc-K + Fam + 0.72 mg/kg | 0.083 | 1457 ± 0.053 | 10.5436 |
| PB(36 mg) | | | |
| Dic-K + Fam + 0.36 mg/kg | 0.083 | 1277 ± 0.075 | 10.382 |
| PB(18 mg) | | | |

### Evaluation of the Results

Table 1 shows the Cmax and Tmax values of diclofenac, diclofenac+famotidine, diclofenac+0.72 mg/kg potassium bicarbonate, disclofenac+famotidine+0.72 mg/kg potassium bicarbonate and diclofenac+famotidine+0.36 mg/kg potassium bicarbonate. As it can be seen there is an evident increase in the Cmax values of diclofenac when it is used in combination with famotidine or potassium bicarbonate or famotidine+potassium bicarbonate. The Cmax of diclofenac when used as a single active ingredient is 1.262 mg/ml, whereas it is 1.457 ng/ml when used in combination with famotidine and 0.72 mg/kg potassium bicarbonate. Similarly, Tmax values in Table 1 also show that combinations of the present invention provide a faster pain relief compared to the diclofenac alone. Tmax of diclofenac when used as a single active ingredient is 0.25 h, whereas it is 0.083 h when used in combination with famotidine and potassium bicarbonate. Combinations of diclofenac+famotidine and diclofenac+potassium bicarbonate also provides efficient pain management compared to diclofenac alone but the combinations of three active ingredients provide the most efficient diclofenac treatment in general by reducing the side effects as well.

In addition to Table 1, Table 2 provides 12 hour AUC values of diclofenac. The combination of diclofenac+famotidine+0.72 mg/kg potassium bicarbonate has the highest AUC value, meaning this combination provides a long duration of pain and inflammation relief. Diclofenac+Famotidine+0.36 mg/kg potassium bicarbonate combination also provides similar results whereas AUC of diclofenac alone shows that when diclofenac is taken alone, duration of pain management is much shorter compared to the combinations.

The results of the study prove the superior treatment modality of the present invention with the surprising effect of longer lasting therapeutically effective blood concentrations (AUC) coupled with the diminished gastro intestinal side effects due to the presence of famotidine in the combinations.

### EXAMPLES

### Example 1

An instant release oral formulation of diclofenac, famotidine and potassium bicarbonate combination.

Table 3 below provides the contents of a composition in the form of a film coated tablet.

TABLE 3
Film Tablet Composition

| Illustrative Film Tablet | Weight (mg)/unit dose |
|---|---|
| Diclofenac potassium | 50 |
| Famotidine | 20 |
| Potassium bicarbonate | 36 |
| Lactose granule | 64 |
| Microcrystalline cellulose | 67 |
| Hydroxypropyl Cellulose | 8 |
| Crospovidone | 10 |
| Colloidal silicon dioxide | 2 |
| Magnesium stearate | 2 |
| Coating based on copovidone with | 7 |
| cellulosic polymers | |

### Example 2

Preparation methods of the pharmaceutical compositions of the present invention

### 1. Preparation Method Wherein the Active Components are not Directly Mixed:

At the first stage, a certain amount of PVP (K-30) is dissolved in purified water. Diclofenac, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator. At the second stage, famotidine is added to the granules obtained in the first stage. Suitable mixing is applied. Final mixture is obtained by the addition of magnesium stearate to the mixture. After the tablets are pressed, they are film-coated.

### 2. Preparation Method Wherein the Active Components are Directly Mixed:

A certain amount of PVP (K-30) is dissolved in purified water. Diclofenac, famotidine, remaining amount of PVP (K-30), lactose, microcrystalline cellulose, sodium starch glycolate and colloidal silicon dioxide are mixed together. The obtained mixture is wet-granulated with the PVP (K-30) solution. Granules so-obtained are dried and pass through the dry granulator. Final mixture is obtained by the addition of magnesium stearate to the mixture. After the tablets are pressed, they are film-coated.

1. A combination comprising:
a) diclofenac or a pharmaceutically acceptable salt thereof,
b) an H2 receptor antagonist, or a pharmaceutically acceptable salt thereof, and
c) an alkali metal carbonate or bicarbonate

2. A combination according to claim 1 wherein the compound (a) is diclofenac sodium or diclofenac potassium.

3. A combination according to claim 1 or 2 wherein the compound (b) is selected from the group consisting of famotidine, pabutidine, lafutidine, loxtidine, nizatidine, roxatidine, tiotidine, niperotidine and oxmetidine, or pharmaceutically acceptable salts thereof.

4. A combination according to any one of claims 1 to 3 wherein the compound (b) is famotidine or a pharmaceutically acceptable salt thereof.

5. A combination according to any one of claims 1 to 4 wherein the compound (c) is selected from the group consisting of potassium bicarbonate, sodium bicarbonate, ammonium bicarbonate and calcium bicarbonate.

6. A combination according to any one of claims 1 to 5 wherein the compound (c) is potassium bicarbonate.

7. A combination according to any one of claims 1 to 6 wherein the amount of diclofenac is between 12.5 to 100 mg.

8. A combination according to any one of claims 1 to 7 wherein the amount of famotidine is between 10 to 60 mg.

9. A combination according to any one of claims 1 to 8 wherein the amount of potassium bicarbonate is between 10 to 120 mg.

10. A combination according to any one of claims 1 to 9 comprising a) diclofenac or a pharmaceutically acceptable salt thereof, b) famotidine or a pharmaceutically acceptable salt thereof, and c) potassium or sodium bicarbonate.

11. A combination according to claim 10 comprising a) between 12.5 to 100 mg of diclofenac or pharmaceutically acceptable salt thereof, b) between 10 to 60 mg of famotidine or pharmaceutically acceptable salt thereof, c) between 10 to 120 mg of potassium bicarbonate.

12. A combination according to any one of claims 1 to 11 for use in the treatment of pain and inflammation.

13. An oral pharmaceutical fixed-dose composition comprising a combination according to claims 1 to 11 and one or more pharmaceutically acceptable excipient(s).

14. A pharmaceutical composition according to claim 13, wherein famotidine and potassium or sodium bicarbonate increases the Cmax of diclofenac by at least 5%.

15. A pharmaceutical composition according to claim 13, wherein famotidine and potassium or sodium bicarbonate creates an AUC at least 10% higher than diclofenac administered alone, within the 12 hours after administration.

16. A pharmaceutical composition according to claim 13, wherein famotidine and potassium or sodium bicarbonate creates a Tmax of less than about 30 minutes for diclofenac.
